(19) United States
(12) Patent Application Publication
(10) Pub. No.: US 2026/0259215 A1
(43) Pub. Date: Sep. 3, 2026

(54) ASSESSING AND TREATING MESOTHELIOMA

(71) Applicant: Mayo Foundation for Medical Education and Research, Rochester, MN (US)

(72) Inventors: Aaron S. Mansfield, Rochester, MN (US); Farhad Kosari, Rochester, MN (US); Mitesh J. Borad, Paradise Valley, AZ (US); Aakash P. Desai, Rochester, MN (US)

(21) Appl. No.: 18/881,830

(22) PCT Filed: Jul. 26, 2023
(86) PCT No.: PCT/US2023/028638

§ 371 Date: Jan. 7, 2025

Related U.S. Application Data
(60) Provisional application No. 63/393,371, filed on Jul. 29, 2022.

(51) Int. Cl. G01N 33/575; A61K 31/282; A61K 31/519; A61K 33/243; A61P 35/00; C07K 16/22

(52) U.S. Cl. CPC G01N 33/57557; A61K 31/282; A61K 31/519; A61K 33/243; A61P 35/00; C07K 16/22; G01N 2333/7051

(57) ABSTRACT

This document relates to methods and materials for assessing and/or treating mammals (e.g., humans) having mesothelioma (e.g., pleural mesothelioma). For example, methods and materials that can be used to determine whether or not a mammal having mesothelioma is likely to respond to a particular cancer treatment (e.g., immunotherapy with one or more immune checkpoint inhibitors) are provided. Methods and materials for treating a mammal (e.g., a human) having mesothelioma (e.g., pleural mesothelioma) where the treatment is selected based, at least in part, on whether or not the mammal is likely to respond to a particular cancer treatment (e.g., immunotherapy with one or more immune checkpoint inhibitors) also are provided.

### CROSS-REFERENCE TO RELATED APPLICATIONS

This application claims the benefit of U.S. Patent Application Ser. No. 63/393,371, filed on Jul. 29, 2022. The disclosure of the prior application is considered part of, and is incorporated by reference in, the disclosure of this application.

### STATEMENT REGARDING FEDERAL FUNDING

This invention was made with government support under CA251923 awarded by the National Institutes of Health. The government has certain rights in the invention.

### SEQUENCE LISTING

This application contains a Sequence Listing that has been submitted electronically as an XML file named “07039-2145WO1.xml.” The XML file, created on Jun. 29, 2023, is 18000 bytes in size. The material in the XML file is hereby incorporated by reference in its entirety.

### TECHNICAL FIELD

This document relates to methods and materials for assessing and/or treating mammals (e.g., humans) having mesothelioma (e.g., pleural mesothelioma). For example, the methods and materials provided herein can be used to determine whether or not a mammal having mesothelioma is likely to respond to a particular cancer treatment (e.g., immunotherapy with one or more immune checkpoint inhibitors). This document also provides methods and materials for treating a mammal (e.g., a human) having mesothelioma (e.g., pleural mesothelioma) where the treatment is selected based, at least in part, on whether or not the mammal is likely to respond to a particular cancer treatment (e.g., immunotherapy with one or more immune checkpoint inhibitors).

### BACKGROUND INFORMATION

Mesothelioma is an aggressive cancer with an average life expectancy of 14 to 22 months. The 5-year relative survival rate for malignant pleural mesothelioma (based on people diagnosed with malignant pleural mesothelioma (MPM) between 2010 and 2016) is only about 10% (American Cancer Society, “Survival Rates for Mesothelioma,” Last Revised: Jan. 21, 2021).

Mesothelioma primarily arises as a result of the exposure to the carcinogen asbestos, although some cases develop after therapeutic radiation, or are inherited due to loss of function mutations in BRCA1 Associated Protein 1 (BAP1) (Carbone et al., CA Cancer J. Clin., 69:402-429 (2019)). Immunotherapy is a frontline treatment option for MPM, with the United States Food and Drug Administration having recently approved a combination therapy with the PD-1 inhibitor nivolumab and the CTLA-4 inhibitor ipilimumab for the treatment of unresectable pleural mesothelioma. However, fewer than half of the patients who receive this regimen respond to it based on modified Response Evaluation Criteria in Solid Tumors (mRECIST) for mesothelioma (Baas et al., Lancet, 397 (10272): 375-386 (2021); and Byrne et al., Ann Oncol., 15:257-260 (2004)).

### SUMMARY

In some cases, this document provides methods and materials for determining whether or not a mammal (e.g., a human) having mesothelioma (e.g., pleural mesothelioma) is likely to respond a particular cancer treatment (e.g., immunotherapy with one or more immune checkpoint inhibitors). For example, a sample (e.g., a blood sample) obtained from a mammal having mesothelioma can be assessed to determine if the mammal is likely to respond a particular cancer treatment (e.g., immunotherapy with one or more immune checkpoint inhibitors) based, at least in part, on whether T cells from the mammal express a T cell receptor (TCR) that includes a CDR3 region of an alpha or beta variable region that includes the amino acid sequence set forth in SEQ ID NO:1 or SEQ ID NO:2.

As demonstrated herein, the presence of T cells having particular TCR CDR3 amino acid sequences (e.g., a CDR3 that includes the amino acid sequence set forth in SEQ ID NO: 1 or SEQ ID NO:2) can indicate that a mammal (e.g., human) having mesothelioma (e.g., pleural mesothelioma) is likely to be responsive to a particular cancer treatment (e.g., immunotherapy with one or more immune checkpoint inhibitors). For example, mammals (e.g., humans) having a mesothelioma and having a detectable level of one or more T cells having a TCR that includes a CDR3 that includes the amino acid sequence set forth in SEQ ID NO: 1 or SEQ ID NO:2 can exhibit improved survival (e.g., as compared to mammals having a mesothelioma and lacking (e.g., lacking a detectable level of) T cells having a TCR that includes a CDR3 that includes the amino acid sequence set forth in SEQ ID NO:1 or SEQ ID NO:2) when treated with one or more immune checkpoint inhibitors.

Having the ability to identify a particular cancer treatment that a mammal (e.g., a human) is most likely to respond to as described herein (e.g., based, at least in part, on whether or not the mammal has T cells having a TCR that includes a CDR3 that includes an amino acid sequence set forth in SEQ ID NO:1 or SEQ ID NO:2) can allow clinicians to provide an individualized approach in selecting cancer treatments. For example, the ability to select a particular cancer treatment based, at least in part, on the likelihood that the patient will respond to that treatment can improve survival (e.g., disease-free survival and/or overall survival) of the patient. For example, the ability to select a particular cancer treatment based, at least in part, on the likelihood that the patient will respond to that treatment can minimize subjecting patients to ineffective treatments. The methods and materials described herein also can be used to carry out antigen discovery and/or to identify future targets for design of adoptive T cell therapies.

In general, one aspect of this document features methods for assessing a mammal having pleural mesothelioma. The methods can include, or consist essentially of, (a) determining if the mammal contains: (i) T cells having a TCR comprising a CDR3 comprising the amino acid sequence set forth in SEQ ID NO:1, or (ii) T cells having a TCR comprising a CDR3 comprising the amino acid sequence set forth in SEQ ID NO:2, (b) classifying the mammal as being likely to respond to an immune checkpoint inhibitor if the mammal contains at least one of (i) and (ii), and (c) classifying the mammal as not being likely to respond to the immune checkpoint inhibitor if the mammal does not contain (i) and (ii). The mammal can be a human. A blood sample obtained from the mammal can be used to determine if the mammal comprises the (i) or (ii). The blood sample can include peripheral blood mononuclear cells (PBMCs). The immune checkpoint inhibitor can be pembrolizumab, nivolumab, cemiplimab, atezolizumab, avelumab, durvalumab, or ipilimumab. The mammal can contain at least one of (i) and (ii). The mammal can contain (i) and (ii). The mammal can contain neither of (i) and (ii).

In another aspect, this document features methods for treating a mammal having pleural mesothelioma. The methods can include, or consist essentially of, (a) determining that the mammal contains: (i) T cells having a TCR comprising a CDR3 comprising the amino acid sequence set forth in SEQ ID NO:1, or (ii) T cells having a TCR comprising a CDR3 comprising the amino acid sequence set forth in SEQ ID NO:2, and (b) administering an immune checkpoint inhibitor to the mammal. The mammal can be a human. A blood sample obtained from the mammal can be used to determine that the mammal contains (i) or (ii). The blood sample can include PBMCs. The mammal can contain each of (i) and (ii). The method also can include administering to the mammal a cancer treatment selected from the group consisting of cisplatin, pemetrexed, surgery, radiation therapy, and ablative therapy.

In another aspect, this document features methods for treating a mammal having pleural mesothelioma. The methods can include, or consist essentially of, (a) determining that the mammal does not contain: (i) T cells having a TCR comprising a CDR3 comprising the amino acid sequence set forth in SEQ ID NO:1, and (ii) T cells having a TCR comprising a CDR3 comprising the amino acid sequence set forth in SEQ ID NO:2, and (b) administering a cancer treatment to the mammal, where the cancer treatment is not an immune checkpoint inhibitor. The mammal can be a human. The blood sample obtained from the mammal can be used to determine that the mammal does not contain the (i) and (ii). The blood sample can include PBMCs. The mesothelioma can be a pleural mesothelioma. The method also can include administering to the mammal a cancer drug selected from the group consisting of cisplatin, pemetrexed, carboplatin, and bevacizumab. The method also can include subjecting the mammal to a treatment selected from the group consisting of surgery, radiation therapy, and ablative therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C show the study design and workflow. FIG. 1A contains a Venn diagram of mesothelioma patient samples, including on-treatment peripheral blood mononuclear cells (PBMC) (bon, n=39), tissue biopsies (ton, n=31), pre-treatment PBMC (bpr, n=49), and tissue biopsy (tpr, n=42). FIG. 1B contains a schematic of a bioinformatics pipeline used to compute TCR clusters. TCR clusters are defined as TCRs with unique DNA or amino acids sequences that share a common antigen. FIG. 1C contains a graph showing the distribution of unique TCR clusters. The dashed line and hashed lines represent median and mean values, respectively.

FIGS. 2A-2C show that normalized counts of TCR membership were correlated with the expression of CD8A (FIG. 2A, left panel) and CD4 (FIG. 2A, right panel). FIG. 2B shows that TCR membership in the on-treatment samples was significantly higher than pre-treatment samples (p=0.017), suggesting that immune checkpoint inhibitors (ICI) increased T cell diversity. FIG. 2C shows that patients with pre-treatment tumors that had TCR cluster membership in the top tertile survived significantly longer than patients with tumors that had TCR cluster membership in the bottom two tertiles.

FIGS. 3A-3C show that the presence of particular TCR clusters can predictor longer survival. FIG. 3A contains a graph showing that patients with pre-treatment PBMCs that had TCR cluster membership in the top tertile survived significantly longer than patients with pre-treatment PBMCs that had TCR cluster membership in the bottom two tertiles. FIG. 3B contains a Venn diagram showing TCR clusters present in healthy controls (hc) and 33 pre-treatment cases with both pre-treatment biopsies (prt) and pre-treatment PBMCs (prb). FIG. 3C contains a graph showing that shared TCR clones between pre-treatment biopsies and PBMCs can predict immunotherapy survival (cox p-value=0.011).

FIGS. 4A-4C show that associations of TCR cluster membership profile with the clinical presentation and overall survival. FIG. 4A contains a heatmap showing TCR cluster profiles in pre-treatment PBMCs. TCR clusters were selected based on having wide membership in mesotheliomas and being exclusive of healthy controls. Rows represent TCR clusters and columns represent patients grouped according to the unsupervised clustering dendrogram on top. Patients to the right of the dashed line were enriched for being among partial responders (chi-square p=0.003) and had longer survival (FIG. 4B) than patients to the left of the dashed line (p=0.038). SD, PR, PD represent stable disease, partial response, and progressive disease, respectively. FIG. 4C contains a graph showing that membership of on-treatment PBMCs in at least one TCR cluster associated with significantly better survival, and that membership in both clusters provided significant survival advantage over membership in only one cluster (p=0.026) or membership in neither cluster (p=0.002).

FIGS. 5A-5B contains Venn diagrams depicting overlaps of TCR cluster membership in pre-treatment (FIG. 5A) and on-treatment (FIG. 5B) samples and healthy controls (hc). prt and prb indicate pre-treatment tissue biopsy and PBMCs, respectively. ont and onb indicate on-treatment tissue biopsy and PBMCs, respectively.

FIG. 6 shows the filtering pipeline for identification of on-treatment TCR clusters with significant survival associations.

FIGS. 7A-7B contains graphs showing immune deconvolution by microenvironment cell populations (MCP)-counter using RNA-seq data depicting increased concentration of T cells (FIG. 7A) and CD8 T-cells (FIG. 7B) in on-treatment compared to pre-treatment PBMCs.

FIGS. 8A-8D show that having membership in a large number of TCR clusters for an on-treatment biopsy or PBMC predicts longer survival. FIG. 8A contains a graph showing that patients with on-treatment tumors that had a TCR cluster membership in the top two tertiles trended toward longer survival than patients with tumors that had TCR cluster membership in the bottom tertile. FIG. 8B contains a graph showing that patients with on-treatment PBMCs that had TCR cluster membership in the top two tertiles survived significantly longer than patients with tumors that had TCR cluster membership in the bottom tertile (p=0.032). FIG. 8C contains a Venn diagram showing the number of TCR clusters in healthy controls and 20 on-treatment cases with both pre-treatment biopsies and PBMCs. FIG. 8D contains a graph showing that shared TCR clones between on-treatment biopsies and PBMCs indicated that patients within the top two tertiles of having shared clones had marginally better survival that patients in the bottom tertile (p=0.071).

FIGS. 9A-9B contain graphs showing that membership in TCR cluster 450844 (SEQ ID NO: 1; FIG. 9A) and TCR cluster 506145 (SEQ ID NO:2; FIG. 9B) was associated with significant improved survival after adjusting for false discovery rate (q-value <0.06).

FIG. 10 contains a list of 100 antigens associated with observed TCRs, including 29 antigens detected exclusively in mesothelioma PBMCs.

### DETAILED DESCRIPTION

This document provides methods and materials for determining whether or not a mammal (e.g., a human) having mesothelioma (e.g., pleural mesothelioma) is likely to respond a particular cancer treatment (e.g., immunotherapy with one or more immune checkpoint inhibitors). For example, a sample (e.g., a blood sample) obtained from a mammal having mesothelioma (e.g., pleural mesothelioma) can be assessed to determine if the mammal is likely to respond a particular cancer treatment (e.g., immunotherapy with one or more immune checkpoint inhibitors) based, at least in part, on whether the mammal has T cells that have a TCR that includes a CDR3 that includes the amino acid sequence set forth in SEQ ID NO:1 or SEQ ID NO:2. In some cases, the methods and materials provided herein also can include administering one or more cancer treatments (e.g., one or more cancer treatments selected based, at least in part, on whether or not the mammal is likely to respond a particular cancer treatment such as immunotherapy with one or more immune checkpoint inhibitors) to a mammal having mesothelioma to treat the mammal.

A mammal (e.g., a human) having mesothelioma (e.g., pleural mesothelioma) can be assessed to determine whether or not the mammal is likely to respond a particular cancer treatment (e.g., immunotherapy with one or more immune checkpoint inhibitors) by detecting the CDR3 sequence present in TCRs of one or more T cells from the mammal. For example, T cells from a mammal (e.g., a human) having mesothelioma (e.g., pleural mesothelioma) can be assessed for the presence or absence of a TCR that includes a CDR3 that includes the amino acid sequence set forth in SEQ ID NO:1 or SEQ ID NO:2. As described herein, the presence or absence of a TCR that includes the amino acid sequence set forth in SEQ ID NO: 1 or SEQ ID NO:2 in one or more T cells obtained from the mammal (e.g., a human) having mesothelioma (e.g., pleural mesothelioma) can be used to determine whether or not that mammal is likely to respond a particular cancer treatment (e.g., immunotherapy with one or more immune checkpoint inhibitors).

Any appropriate mammal having mesothelioma (e.g., pleural mesothelioma) can be assessed and/or treated as described herein. Examples of mammals that can have mesothelioma and can be assessed and/or treated as described herein include, without limitation, humans, non-human primates (e.g., monkeys), dogs, cats, horses, cows, pigs, sheep, mice, and rats. In some cases, a mammal having mesothelioma that can be assessed and/or treated as described herein can be a mammal that was exposed to asbestos. In some cases, a mammal having mesothelioma that can be assessed and/or treated as described herein can be a mammal that was administered therapeutic radiation. In some cases, a mammal having mesothelioma that can be assessed and/or treated as described herein can be a mammal that has one or more loss of function mutations in one or both copies of the mammal's BAP1 gene.

When assessing and/or treating a mammal (e.g., a human) having mesothelioma as described herein, the mesothelioma can be any type of mesothelioma. A mesothelioma can be any stage of mesothelioma (e.g., stage I, stage II, stage III, or stage IV). A mesothelioma can involve any mesothelium tissue. For example, a mesothelioma can involve a tissue that surrounds the lungs (lung lining), tissue that surrounds the abdomen (abdominal lining), tissue that surrounds the heart (heart sac), and/or the tissue that surrounds the testes (tunica vaginalis). A mesothelioma can include any cell type. For example, a mesothelioma can include epithelioid cells, sarcomatoid cells, biphasic cells, dermoplastic cells, and/or histiocytoid cells. Examples of types of mesothelioma a mammal (e.g., a human) being assessed and/or treated as described herein can have include, without limitation, pleural mesothelioma (e.g., MPM), peritoneal mesothelioma, pericardial mesothelioma, mesothelioma of the tunica vaginalis. In some cases, a mesothelioma can be a primary cancer (e.g., a localized primary cancer). In some cases, a mesothelioma can have metastasized. In some cases, a mesothelioma can be an unresectable mesothelioma.

In some cases, the methods described herein can include identifying a mammal (e.g., a human) as having mesothelioma (e.g., pleural mesothelioma). Any appropriate method can be used to identify a mammal as having mesothelioma. For example, physical examination (e.g., to check for lumps), imaging techniques (e.g., X-rays such as such as a chest X-ray, computerized tomography (CT) scanning, magnetic resonance imaging (MRI), and positron emission tomography (PET)), laboratory examination (e.g., of a sample such as a tissue sample obtained by biopsy or surgical resection), and/or visualization techniques (e.g., bronchoscopy) can be used to identify a mammal (e.g., a human) as having mesothelioma.

In some cases, a sample (e.g., a blood sample) obtained from a mammal (e.g., a human) having mesothelioma (e.g., pleural mesothelioma) can be assessed to determine whether or not the mammal has T cells that have a TCR that includes a CDR3 that includes the amino acid sequence set forth in SEQ ID NO:1. The amino acid sequence set forth in SEQ ID NO:1 is as follows, and the amino acid sequences of SEQ ID NO: 1 fall within cluster 450844:

SEQ ID NO: 1 = CASSYS(N/S/D/E)(R/Q)D(H/Y/F)GYTF

In some cases, a sample (e.g., a blood sample) obtained from a mammal (e.g., a human) having mesothelioma (e.g., pleural mesothelioma) can be assessed to determine whether or not the mammal has T cells that have a TCR that includes a CDR3 that includes the amino acid sequence set forth in SEQ ID NO:2. The amino acid sequence set forth in SEQ ID NO:2 is as follows, and the amino acid sequences of SEQ ID NO:2 fall within cluster 506145:

SEQ ID NO: 2 = CS(A/V)(S/T)GTG(N/S/A)(N/H)QPQHF

In some cases, a sample (e.g., a blood sample) obtained from a mammal (e.g., a human) having mesothelioma (e.g., pleural mesothelioma) can be assessed to determine whether or not the mammal has T cells that have a TCR that includes a CDR3 that includes the amino acid sequence set forth in SEQ ID NO:1 or SEQ ID NO:2. For example, a mammal having mesothelioma (e.g., pleural mesothelioma) and identified as having a detectable level of one or more T cells that have a TCR that includes a CDR3 that includes the amino acid sequence set forth in SEQ ID NO:1 can be identified as being a mammal likely to respond a particular cancer treatment (e.g., immunotherapy with one or more immune checkpoint inhibitors). In some cases, a mammal having mesothelioma (e.g., pleural mesothelioma) and identified as having a detectable level of one or more T cells that have a TCR that includes a CDR3 that includes the amino acid sequence set forth in SEQ ID NO:2 can be identified as being a mammal likely to respond a particular cancer treatment (e.g., immunotherapy with one or more immune checkpoint inhibitors).

In some cases, a sample (e.g., a blood sample) obtained from a mammal (e.g., a human) having mesothelioma (e.g., pleural mesothelioma) can be assessed to identify the mammal as having (a) one or more T cells (e.g., a detectable level of T cells) that have a TCR that includes a CDR3 that includes the amino acid sequence set forth in a first one of SEQ ID NO: 1 or SEQ ID NO:2 and (b) one or more T cells (e.g., a detectable level of T cells) that have a TCR that includes a CDR3 that includes the amino acid sequence set forth in a second one of SEQ ID NO: 1 or SEQ ID NO:2. For example, a mammal having mesothelioma (e.g., pleural mesothelioma) and identified as having a detectable level of (a) one or more T cells that have a TCR that includes a CDR3 that includes the amino acid sequence set forth in SEQ ID NO: 1 and (b) one or more T cells that have a TCR that includes a CDR3 that includes the amino acid sequence set forth in SEQ ID NO:2 can be identified as being a mammal likely to respond a particular cancer treatment (e.g., immunotherapy with one or more immune checkpoint inhibitors). In another example, a mammal having mesothelioma (e.g., pleural mesothelioma) and identified as having a detectable level of (a) one or more T cells that have a TCR that includes a CDR3 that includes the amino acid sequence set forth in SEQ ID NO:2 and (b) one or more T cells that have a TCR that includes a CDR3 that includes the amino acid sequence set forth in SEQ ID NO: 1 can be identified as being a mammal likely to respond a particular cancer treatment (e.g., immunotherapy with one or more immune checkpoint inhibitors). In some cases, a sample (e.g., a blood sample) obtained from a mammal (e.g., a human) having mesothelioma (e.g., pleural mesothelioma) can be assessed to identify the mammal as having (a) one or more T cells (e.g., a detectable level of T cells) that have a TCR that includes a CDR3 that includes the amino acid sequence set forth in SEQ ID NO: 1, and (b) one or more T cells (e.g., a detectable level of T cells) that have a TCR that includes a CDR3 that includes the amino acid sequence set forth in SEQ ID NO:2. For example, a mammal having mesothelioma (e.g., pleural mesothelioma) and identified as having a detectable level of (a) one or more T cells that have a TCR that includes a CDR3 that includes the amino acid sequence set forth in SEQ ID NO: 1, and (b) one or more T cells that have a TCR that includes a CDR3 that includes the amino acid sequence set forth in SEQ ID NO: 2 can be identified as being a mammal likely to respond a particular cancer treatment (e.g., immunotherapy with one or more immune checkpoint inhibitors).

Any appropriate T cell-containing sample from a mammal (e.g., a human) having mesothelioma (e.g., pleural mesothelioma) can be assessed as described herein (e.g., to determine whether or not the mammal has one or more T cells that have a TCR that includes a CDR3 that includes the amino acid sequence set forth in SEQ ID NO:1 or SEQ ID NO:2). In some cases, a sample can be a biological sample. In some cases, a sample (e.g., a blood sample such as a whole blood sample) can include one or more peripheral blood mononuclear cells (PBMCs). In some cases, a sample can contain one or more cancer cells (e.g., one or more mesothelioma cells). In some cases, a sample can contain one or more biological molecules (e.g., nucleic acids such as DNA and RNA, polypeptides, carbohydrates, lipids, hormones, and/or metabolites). In some cases, a sample can contain one or more lymphocytes (e.g., lymphocytes having TCR sequences). Examples of types of samples that can be assessed as described herein include, without limitation, fluid samples (e.g., whole blood, urine, and saliva), tissue samples (e.g., pleural tissues, mesothelium tissues, or tunica vaginalis tissues), and cellular samples (e.g., buccal swabs). A sample can be a fresh sample or a fixed sample (e.g., a formaldehyde-fixed sample or a formalin-fixed sample). In some cases, one or more biological molecules can be isolated from a sample. For example, nucleic acid can be isolated from a sample and can be assessed as described herein. For example, polypeptides can be isolated from a sample and can be assessed as described herein.

Any appropriate method can be used to determine whether T cells have a TCR that includes a CDR3 that includes the amino acid sequence set forth in SEQ ID NO:1 or SEQ ID NO: 2. In some cases, the CDR3 sequence of a TCR can be determined by sequencing the mRNA encoding the TCR. Examples of methods that can be used to sequence mRNA encoding a TCR to detect TCRs that include a CDR3 that includes the amino acid sequence set forth in SEQ ID NO:1 or SEQ ID NO:2 include, without limitation, Sanger sequencing, next-generation sequencing (NGS), immunosequencing, and bioinformatics methods (e.g., bioinformatics methods that can extract TCR sequences from RNA-sequencing data such as MIXCR, CATT, and TRUST). In some cases, the CDR3 sequence of a TCR can be determined by sequencing the TCR alpha or beta polypeptide. Examples of methods that can be used to sequence polypeptides include, without limitation, mass spectrometry techniques (e.g., proteomics-based mass spectrometry assays or targeted quantification-based mass spectrometry assays). In some cases, the CDR3 sequence of a TCR can be assessed as described in Example 1.

In some cases, the presence of T cells that have a TCR that includes a CDR3 that includes the amino acid sequence set forth in SEQ ID NO:1 or SEQ ID NO:2 can indicate that a mammal is likely to respond a particular cancer treatment (e.g., immunotherapy with one or more immune checkpoint inhibitors). For example, a mammal (e.g., a human) having mesothelioma (e.g., pleural mesothelioma) can be identified as being likely to respond a particular cancer treatment (e.g., immunotherapy with one or more immune checkpoint inhibitors) based, at least in part, on the mammal having one or more T cells having a TCR that includes a CDR3 that includes the amino acid sequence set forth in SEQ ID NO:1 or SEQ ID NO:2.

In some cases, a mammal lacking detectable levels of any T cells that have a TCR that includes a CDR3 that includes the amino acid sequence set forth in SEQ ID NO: 1 or SEQ ID NO:2 can indicate that the mammal is not likely to respond a particular cancer treatment (e.g., immunotherapy with one or more immune checkpoint inhibitors). For example, a mammal (e.g., a human) having mesothelioma (e.g., pleural mesothelioma) can be identified as not likely to respond a particular cancer treatment (e.g., immunotherapy with one or more immune checkpoint inhibitors) based, at least in part, on the mammal lacking T cells (e.g., lacking detectable levels of T cells) that have a TCR that includes a CDR3 that includes the amino acid sequence set forth in SEQ ID NO:1 or SEQ ID NO:2.

In some cases, a mammal (e.g., a human) having mesothelioma (e.g., pleural mesothelioma) and identified as being likely to respond a particular cancer treatment (e.g., immunotherapy with one or more immune checkpoint inhibitors) as described herein (e.g., based, at least in part, the mammal having one or more T cells that have a TCR that includes a CDR3 that includes the amino acid sequence set forth in SEQ ID NO:1 or SEQ ID NO:2) can be selected for treatment with one or more immunotherapies (e.g., one or more immune checkpoint inhibitors). For example, a mammal having mesothelioma (e.g., pleural mesothelioma) and identified as having one or more T cells that have a TCR that includes a CDR3 that includes the amino acid sequence set forth in SEQ ID NO: 1 or SEQ ID NO:2 can be selected to receive one or more (e.g., one, two, three, four, five, or more) immunotherapies.

In some cases, a mammal (e.g., a human) having mesothelioma (e.g., pleural mesothelioma) and identified as not being likely to respond to a particular cancer treatment (e.g., immunotherapy with one or more immune checkpoint inhibitors) as described herein (e.g., based, at least in part, on the mammal lacking (e.g., lacking detectable levels of) any T cells that have a TCR that includes a CDR3 that includes the amino acid sequence set forth in SEQ ID NO:1 or SEQ ID NO:2 can be selected for treatment with one or more alternative cancer treatments (e.g., one or more cancer treatments that are not an immunotherapy). For example, a mammal having mesothelioma (e.g., pleural mesothelioma) and identified as lacking (or as lacking detectable levels of) any T cells that have a TCR that includes a CDR3 that includes the amino acid sequence set forth in SEQ ID NO:1 or SEQ ID NO:2 can be selected to receive one or more (e.g., one, two, three, four, five, or more) alternative cancer treatments.

This document also provides methods for treating a mammal (e.g., a human) having mesothelioma (e.g., pleural mesothelioma). In some cases, a mammal (e.g., a human) having mesothelioma (e.g., pleural mesothelioma) and assessed as described herein (e.g., to determine whether or not the mammal is likely to respond a particular cancer treatment such as immunotherapy with one or more immune checkpoint inhibitors based, at least in part, the mammal having one or more T cells (or lacking any T cells) having a TCR that includes a CDR3 that includes the amino acid sequence set forth in SEQ ID NO:1 or SEQ ID NO:2) can be administered or instructed to self-administer one or more (e.g., one, two, three, four, five, or more) cancer treatments, where the one or more cancer treatments are effective to treat the cancer within the mammal. For example, a mammal having mesothelioma (e.g., pleural mesothelioma) can be administered or instructed to self-administer one or more cancer treatments selected based, at least in part, on whether or not the mammal is likely to respond to a particular cancer treatment such as immunotherapy with one or more immune checkpoint inhibitors as described herein.

In general, a cancer treatment for mesothelioma (e.g., pleural mesothelioma) can include any appropriate mesothelioma cancer treatment. In some cases, a cancer treatment can include administering one or more cancer drugs (e.g., chemotherapeutic agents, targeted cancer drugs, immunotherapy drugs, corticosteroids, and hormones) to a mammal in need thereof. Examples of cancer drugs that can be administered to a mammal having mesothelioma (e.g., pleural mesothelioma) can include, without limitation, pembrolizumab (e.g., KEYTRUDA®), nivolumab (e.g., OPDIVO®), cemiplimab (e.g., LIBTAYO®), atezolizumab (e.g., TECENTRIQ®), avelumab (e.g., Bavencio®), durvalumab (e.g., IMFINZI®), ipilimumab (e.g., YERVOY®), cisplatin (e.g., PLATINOL®), pemetrexed (e.g., Alimta® and PEMFEXY™), carboplatin, gemcitabine, vinorelbine, ramucirumab, bevacizumab, and combinations thereof. In some cases, a cancer treatment for mesothelioma (e.g., pleural mesothelioma) can include surgery and other medical interventions. Examples of surgeries and other medical interventions that can be performed on a mammal having mesothelioma (e.g., pleural mesothelioma) to treat the mammal include, without limitation, surgery (e.g., to decrease fluid buildup in the chest (pleurodesis), to remove the tissue around the lungs (pleurectomy), and to remove a lung and the surrounding tissue, for peritoneal mesothelioma) such as cytoreductive surgery, radiation therapy, and ablative therapy (e.g., cryoablation and radiofrequency ablation).

When treating a mammal (e.g., a human) having mesothelioma (e.g., pleural mesothelioma) and identified as being likely to respond a particular cancer treatment (e.g., immunotherapy with one or more immune checkpoint inhibitors) as described herein, the mammal can be administered or instructed to self-administer one or more (e.g., one, two, three, four, five, or more) immunotherapies (e.g., one or more immune checkpoint inhibitors). For example, a mammal having mesothelioma (e.g., pleural mesothelioma) and identified as having one or more T cells having a TCR that includes a CDR3 that includes the amino acid sequence set forth in SEQ ID NO:1 or SEQ ID NO:2 can be administered or instructed to self-administer one or more immune checkpoint inhibitors. In some cases, an immune checkpoint inhibitor can inhibit (e.g., can reduce) PD-1 signaling (e.g., can inhibit polypeptide expression or polypeptide activity of a programmed cell death protein 1 (PD-1) receptor polypeptide or can inhibit polypeptide expression or polypeptide activity of a programmed death-ligand 1 (PD-L1) polypeptide). In some cases, an immune checkpoint inhibitor can inhibit (e.g., can reduce) cytotoxic T-lymphocyte-associated protein 4 (CTLA-4) signaling (e.g., can inhibit polypeptide expression or polypeptide activity of a CTLA-4 polypeptide). Examples of immune checkpoint inhibitors that can be administered to a mammal (e.g., a human) having mesothelioma (e.g., pleural mesothelioma) and having one or more T cells having a TCR that includes a CDR3 that includes the amino acid sequence set forth in SEQ ID NO:1 or SEQ ID NO:2 include, without limitation, pembrolizumab (e.g., KEYTRUDA®), nivolumab (e.g., OPDIVO®), cemiplimab (e.g., LIBTAYO®), atezolizumab (e.g., TECENTRIQ®), avelumab (e.g., Bavencio®), durvalumab (e.g., IMFINZI®), and ipilimumab (e.g., YERVOY®).

In some cases, when treating a mammal (e.g., a human) having mesothelioma (e.g., pleural mesothelioma) and identified as being likely to respond a particular cancer treatment (e.g., immunotherapy with one or more immune checkpoint inhibitors) as described herein, the one or more (e.g., one, two, three, four, five, or more) immunotherapies (e.g., one or more immune checkpoint inhibitors) can be the sole active agent(s) administered to the mammal to treat the mesothelioma.

In some cases, when treating a mammal (e.g., a human) having mesothelioma (e.g., pleural mesothelioma) and identified as being likely to respond a particular cancer treatment (e.g., immunotherapy with one or more immune checkpoint inhibitors) as described herein, the one or more (e.g., one, two, three, four, five, or more) immunotherapies (e.g., one or more immune checkpoint inhibitors) can be administered to the mammal together with one or more additional agents/therapies used to treat mesothelioma. Examples of anti-cancer treatments that can be administered to a mammal (e.g., a human) having mesothelioma together with one or more immunotherapies (e.g., one or more immune checkpoint inhibitors) include, without limitation, administering one or more chemotherapy drugs such as cisplatin (e.g., PLATINOL®), pemetrexed (e.g., Alimta® and PEMFEXY™), carboplatin, bevacizumab, and any combinations thereof. In cases where one or more immunotherapies (e.g., one or more immune checkpoint inhibitors) are used in combination with additional agents used to treat cancer, the one or more additional agents can be administered at the same time (e.g., in a single composition containing both one or more immunotherapies and the one or more additional agents) or independently. For example, one or more immunotherapies (e.g., one or more immune checkpoint inhibitors) can be administered first, and the one or more additional agents administered second, or vice versa. Examples of therapies that can be used to treat cancer include, without limitation, surgery (e.g., to decrease fluid buildup in the chest (pleurodesis), to remove the tissue around the lungs (pleurectomy), and to remove a lung and the surrounding tissue, for peritoneal mesothelioma) such as cytoreductive surgery, radiation therapy, and ablative therapy (e.g., cryoablation and radiofrequency ablation). In cases where one or more immunotherapies (e.g., one or more immune checkpoint inhibitors) are used in combination with one or more additional therapies used to treat mesothelioma, the one or more additional therapies can be performed at the same time or independently of the administration of the one or more immunotherapies. For example, one or more immunotherapies (e.g., one or more immune checkpoint inhibitors) can be administered before, during, or after the one or more additional therapies are performed.

When treating a mammal (e.g., a human) having mesothelioma (e.g., pleural mesothelioma) and identified as not being likely to respond a particular cancer treatment (e.g., immunotherapy with one or more immune checkpoint inhibitors) as described herein, the mammal can be administered or instructed to self-administer one or more alternative cancer treatments (e.g., one or more cancer treatments that are not an immunotherapy). For example, a mammal having mesothelioma (e.g., pleural mesothelioma) and identified as lacking any T cells having a TCR that includes a CDR3 that includes the amino acid sequence set forth in SEQ ID NO: 1 or SEQ ID NO:2 can be administered or instructed to self-administer one or more alternative cancer treatments. Examples of alternative cancer treatments that can be administered to a mammal (e.g., a human) having mesothelioma (e.g., pleural mesothelioma) and identified as lacking any T cells having a TCR that includes a CDR3 that includes the amino acid sequence set forth in SEQ ID NO:1 or SEQ ID NO:2 to treat the mammal include, without limitation, administering one or more chemotherapy drugs (e.g., cisplatin (e.g., PLATINOL®), pemetrexed (e.g., Alimta® and PEMFEXY™), carboplatin, gemcitabine, vinorelbine, ramucirumab, and/or bevacizumab), surgery (e.g., to decrease fluid buildup in the chest (pleurodesis), to remove the tissue around the lungs (pleurectomy), and to remove a lung and the surrounding tissue, for peritoneal mesothelioma) such as cytoreductive surgery, radiation therapy, and ablative therapy (e.g., cryoablation and radiofrequency ablation).

In some cases, a mammal (e.g., a human) having mesothelioma (e.g., pleural mesothelioma) and identified as not being likely to respond a particular cancer treatment (e.g., immunotherapy with one or more immune checkpoint inhibitors) as described herein is not administered an immunotherapy (e.g., an immune checkpoint inhibitor).

In some cases, when treating a mammal (e.g., a human) having mesothelioma (e.g., pleural mesothelioma) as described herein, the treatment can be effective to treat the mesothelioma. For example, the number of cancer cells present within a mammal can be reduced using the methods and materials described herein. In some cases, the methods and materials described herein can be used to reduce the number of cancer cells present within a mammal having mesothelioma by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having mesothelioma by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having mesothelioma (e.g., pleural mesothelioma) as described herein, the treatment can be effective to improve survival of the mammal. For example, the methods and materials described herein can be used to improve disease-free survival (e.g., relapse-free survival). For example, the methods and materials described herein can be used to improve overall survival. For example, the methods and materials described herein can be used to improve the survival of a mammal having mesothelioma by, for example, 10, 20, 30, 40, 50, 60, 70, 80, 90, 95, or more percent. For example, the methods and materials described herein can be used to improve the survival of a mammal having mesothelioma by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having mesothelioma (e.g., pleural mesothelioma) as described herein, the treatment can be effective to reduce or eliminate one or more symptoms of the mesothelioma. Examples of symptoms mesothelioma that can be reduced or eliminated using the methods and materials described herein can include, without limitation, chest pain, painful coughing, shortness of breath, unusual lumps of tissue under the skin on your chest, unexplained weight loss, abdominal pain, abdominal swelling, nausea, unexplained weight loss, difficulty breathing, and chest pains. For example, the methods and materials described herein can be used to reduce one or more symptoms of mesothelioma within a mammal having mesothelioma by, for example, 10, 20, 30, 40, 50, 60, 70, 80, 90, 95, or more percent.

In some cases, when treating a mammal (e.g., a human) having mesothelioma (e.g., pleural mesothelioma) as described herein, the treatment can be effective to reduce or eliminate one or more complications associated with the mesothelioma. Examples of complications of mesothelioma that can be reduced or eliminated using the methods and materials described herein include, without limitation, difficulty breathing, chest pain, difficulty swallowing, and pain (e.g., caused by pressure on the nerves and spinal cord), accumulation of fluid in the chest (pleural effusion). For example, the methods and materials described herein can be used to reduce one or more complications associated with mesothelioma within a mammal having mesothelioma by, for example, 10, 20, 30, 40, 50, 60, 70, 80, 90, 95, or more percent.

In some cases, a course of treatment, the number of cancer cells present within a mammal, and/or the severity of one or more symptoms related to the mesothelioma (e.g., pleural mesothelioma) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention described in the claims.

### EXAMPLES

### Example 1: Dynamics and Survival Associations of T Cell Clusters in Patients with Pleural Mesothelioma Treated with Immunotherapy

T-cell receptors (TCR) determine the antigen specificity of T cells. TCR-sequencing (TCRseq) can identify unique TCR sequences that recognize the same antigen due to similarities in the complementarity-determining region 3 (CDR3) sequences. This Example describes the identification of TCR CDR3 clusters having predictive significance in patients with MPM and treated with ICI.

### Methods

### Patients and Specimens:

A total of 68 patients with pleural mesothelioma were treated with the PD-1 inhibitor nivolumab alone or in combination with the CTLA-4 inhibitor ipilimumab on the NivoMes (n=34) (Quispel-Janssen et al., J. Thoracic Onc. 13:1569-1576 (2018)) and INITIATE (n=34) (Disselhorst et al., Lancet Resp. Med. 7:260-270 (2019)) clinical trials. Tumor biopsies were obtained from patients prior to treatment with nivolumab after previous treatment with platinum-based chemotherapy, or prior to treatment with nivolumab and ipilimumab after previous treatment with platinum-based chemotherapy (n=42 pre-treatment biopsy specimens). Tumor biopsies were also collected after six weeks of treatment from most of these patients (n=31). RNA from the tumor biopsies was purified using the AllPrep DNA/RNA/miRNA Universal kit (Qiagen, #80224) following the instructions provided by the manufacturer. The buffer included β-mercaptoethanol for the specimens obtained from NCT02497508, and dithiothreitol for the ones obtained from NCT03048474. Otherwise, there were no differences in the handling of the specimens or nucleic acid purification. Peripheral blood mononuclear cells (PBMCs) were drawn in EDTA tubes and frozen prior to initiation of immunotherapy, and six weeks after the start of therapy for a total of 49 pre-treatment and 39 on-treatment PBMCs for TCRseq. The DNA was purified from the PBMCs and prepared for TCRseq per instructions from Adaptive Biotechnologies. The responses to treatment with ICI that were determined by modified pleural RECIST (Quispel-Janssen et al., J. Thoracic Onc. 13:1569-1576 (2018) and Disselhorst et al., Lancet Resp. Med. 7:260-270 (2019)) were also used for analyses.

### Processing of RNA and TCR Sequencing Data:

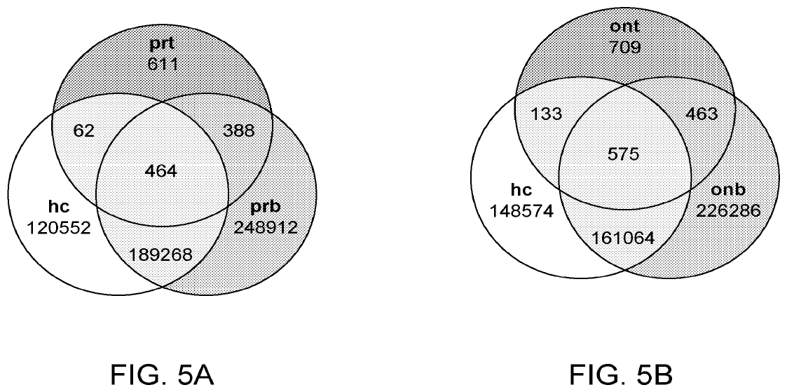

RNA fastq files from tumor biopsies were processed by TRUST4 python program using the default settings to identify TCR CDR3 amino acid sequences and the corresponding variable chains in each tumor biopsy. These tissue-based TCR sequences were pooled with the TCR sequences found in PBMCs as determined by TCRseq from the trial participants, and with publically available TCR sequences identified in PBMCs from healthy controls. The TCR sequences from healthy controls without cancer were obtained from an investigation on the effects of cytomegalovirus exposure history on the T cell repertoire (Emerson et al., Nat. Genetics 49:659-665 (2017)) and were downloaded from the Adaptive Biotechnologies immuneACCESS platform. The analysis of the healthy control TCRs clones was restricted to those with ≥5 copies since cancer-specific low abundant naïve T cells can be present in healthy individuals (Jenkins et al., J. Immuno. 188:4135-4140 (2012)). Altogether, there were approximately 12,200, 4.2 million, and 2.1 million CDR3 amino acid sequences and corresponding variable chains in mesothelioma tumor biopsies, PBMCs of patients with mesothelioma, and healthy controls, respectively. After processing these data with the GIANA python program using default settings, 585,920 computationally derived TCR clusters were identified (FIG. 5). To maximize identification of TCR that were possibly specific to MPM, TCR clusters that were also identified in healthy controls were excluded. Additionally, TCR clusters were normalized by sequencing depths (mapped reads) and the number of PBMCs used for TCRseq, respectively, for comparisons of TCR clusters in tissue biopsies and PBMCs.

### Unsupervised PBMC Clustering and Heatmap:

There were 29 TCR clusters that were identified in at least 12 pre-treatment PBMC samples. These commonly found TCR clusters were used to investigate the similarity in pre-treatment PBMC profiles by unsupervised clustering using the “cosine” similarity function in the lsa package in R with the “ward.D” clustering method. The heatmap function in ‘stats’ package was used to generate the heatmap in R.

Associations of Individual PBMC TCRs with Treatment Outcome:

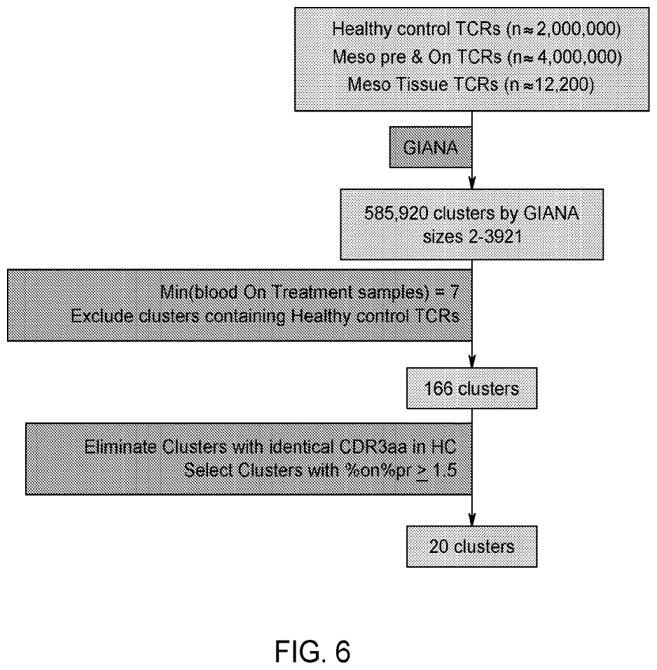

The study aimed to determine whether any TCR cluster was significantly associated with survival upon treatment with ICI. Associations were based on log rank p values determined by the ‘coxph’ function in the “survival” package in R. The focus was limited to TCR clusters identified in at least 7 individual patients. The data did not identify any pre-treatment PBMCs with significant association with overall survival after correcting for the false discover rate (FDR). In the on-treatment samples, there were 166 clusters from at least 7 individuals (FIG. 6). Clusters were eliminated that (1) contained identical CDR3 amino acid sequences in healthy controls even if with a different variable chain, and (2) were not more prevalent in on-treatment than pre-treatment PBMCs because any TCR cluster associated with treatment response would likely expand upon treatment. This search identified two clusters that were associated with overall survival following treatment with immunotherapy with a q-value <0.056 calculated by “qvalue” package. To plot multiple KM plots, km.coxph.plot function was used in the “survcomp” package in R.

### Normalized Tumor Biopsy Gene Expression Matrix and Immune Deconvolution:

Mapping of the RNA-seq data and estimations of gene expression counts in each tumor biopsy sample were performed with the MAP-RSeq pipeline developed by the Mayo Clinic Bioinformatics Core.24 Raw “count” files were processed by the “edgeR” package to generate log 2 normalized gene expression values. Estimations of T cells and other immune cells in tumor biopsies were performed with the “immunedeconv” package in R.

### Results

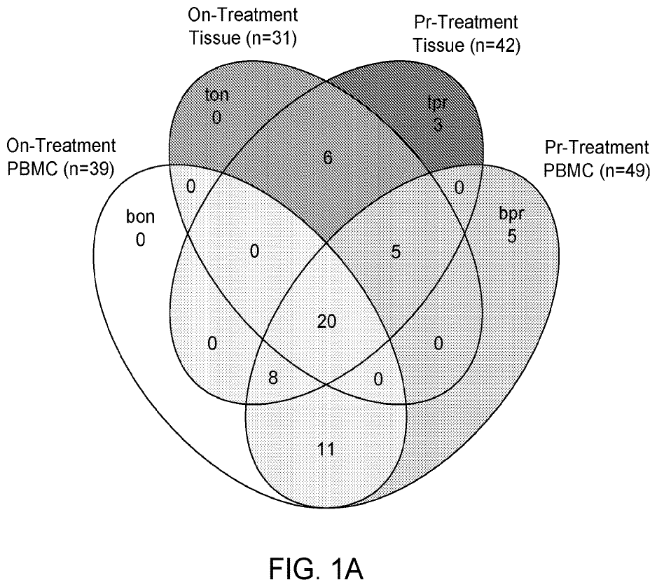

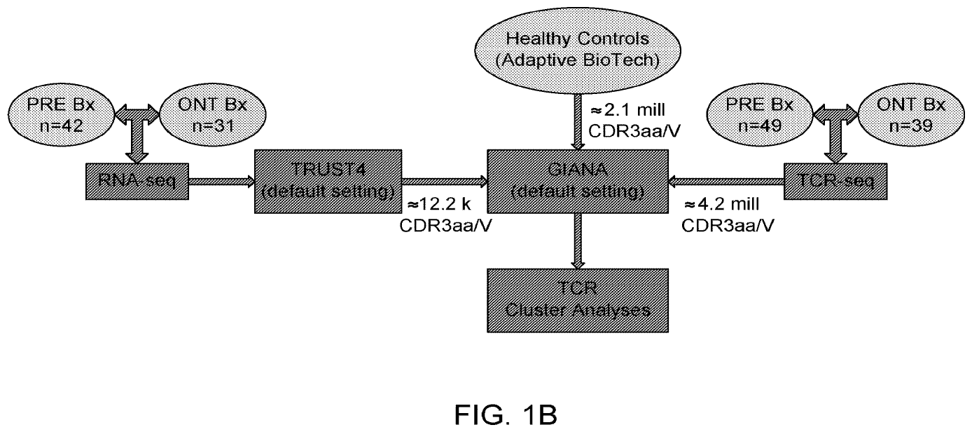

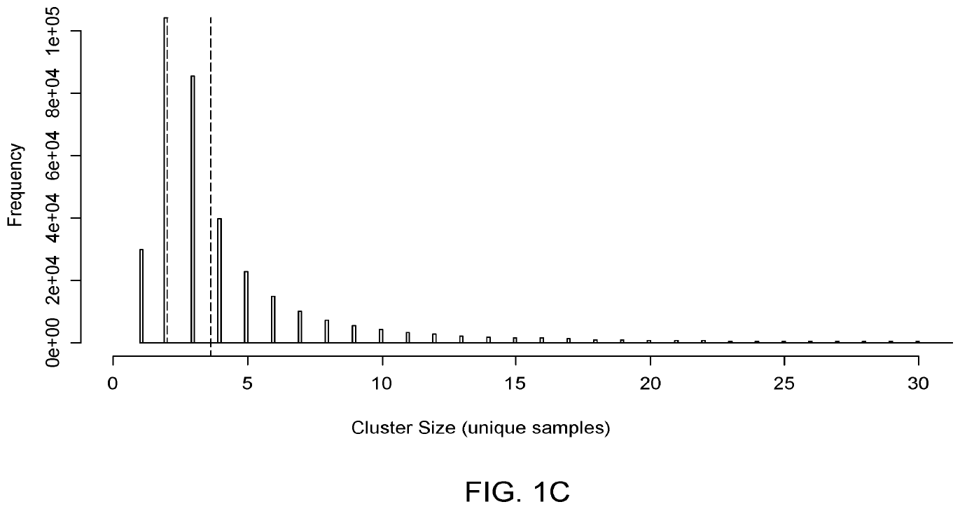

The aim of this study was to investigate the relationship between computationally derived TCR clusters from tissue biopsies and PBMCs with survival with ICI therapy (FIGS. 1A-C, Table 1). TCR sequences were analyzed from 3 sources including (1) healthy controls (n=2.1 million), (2) PBMCs from patients with MPM obtained just prior to treatment or six weeks after treatment (n≈4.2 million), and (3) in pre-treatment or on treatment biopsies from these patients (n=12,200). These TCR sequences were analyzed by the GIANA TCR clustering program which identified close to 586,000 TCR clusters with predicted shared antigen specificity and each containing an average of 4.6 individual TCR sequences. The median and the mean of unique samples in TCR clusters were 2 and 3.66, respectively (FIG. 1C). Venn diagrams of overlapping clusters between MPM tumor biopsies, MPM PBMCs, and healthy controls from pre-treatment or on treatment samples are shown in FIG. 5. In all subsequent analyses, clusters were examined that were not identified in healthy controls to focus on mesothelioma-associated TCRs.

TABLE 1
Patient characteristics

| CHARACTERISTICS | PATIENTS (N = 44) | |
|---|---|---|
| SEX, N (%) | | |
| MALE | 37 | (84%) |
| FEMALE | 7 | (16%) |
| HISTOLOGY, N (%) | | |
| EPITHELIOID | 38 | (86%) |
| BIPHASIC | 3 | (7%) |
| SARCOMATOID | 3 | (7%) |
| TREATMENT, N (%) | | |
| NIVOLUMAB | 24 | (55%) |
| NIVOLUMAB, IPILIMUMAB | 20 | (45%) |
| LINE OF THERAPY, N (%) | | |
| 2 | 38 | (86%) |
| ≥3 | 6 | (14%) |
| AGE, MEDIAN (RANGE) | 66 | (47-81) |

Impact of Pre-Treatment TCR Clusters on Survival with Immunotherapy:

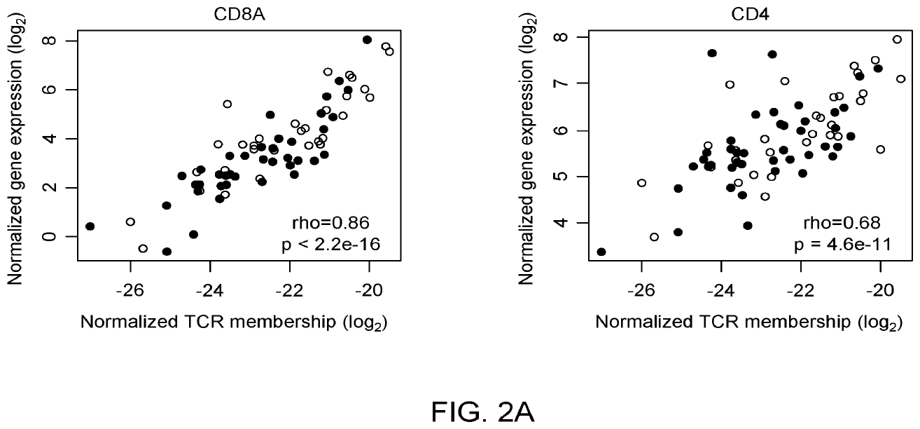

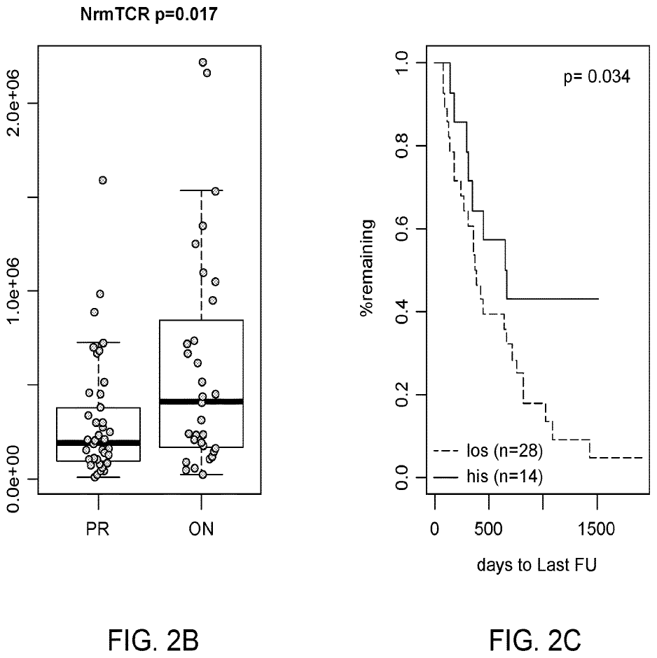

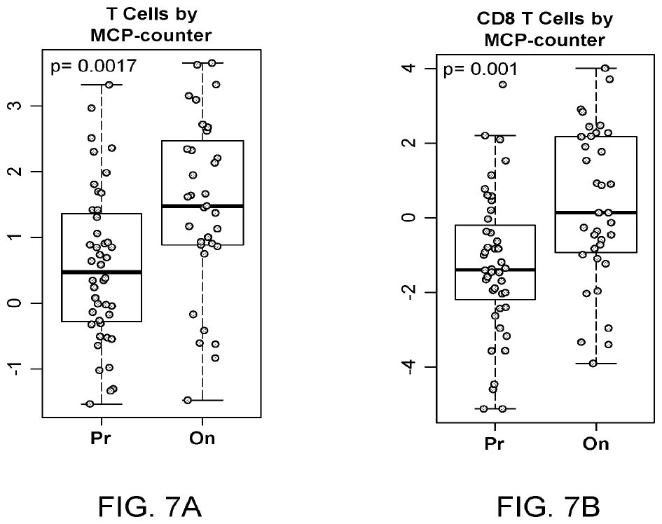

The normalized count of TCR clusters identified in tumor tissue was highly correlated with the expression of CD8A and CD4 genes (FIGS. 2A-B). In other words, tumors with high TCR cluster counts had high CD8A and CD4 expression in tumor biopsies by RNA-seq. Additionally, in tumor biopsies the number of TCR clusters detected in on-treatment samples were significantly higher than in pre-treatment samples (p=0.017, FIG. 2C). This finding was consistent with the immune deconvolution analyses that found greater T cell accumulation in biopsy specimens following treatment with ICI compared to the pre-treatment specimens (FIG. 7). The data suggested that ICI increased T cell clonal diversity (based on the increase in T cell clusters) and trafficking to mesotheliomas. Notably, patients with pre-treatment tumor biopsies with normalized TCR cluster membership in the top tertile had significantly better survival than patients with TCR clusters in the bottom two tertiles (p=0.034, HR=0.44, 95% CI=0.200-0.986, FIG. 2D).

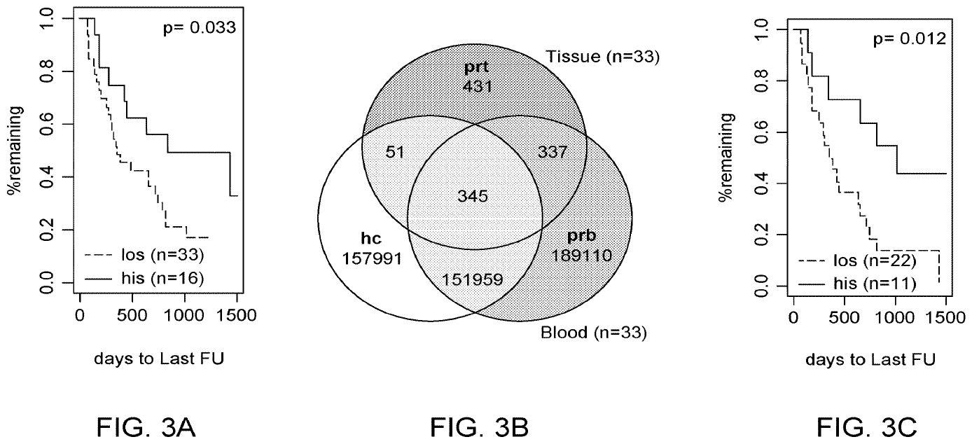

Similarly, patients with pre-treatment PBMCs with the top tertile of TCR cluster counts had significantly longer survival than patients with TCR clusters in the bottom two tertiles (p=0.033, HR=0.41, 95% CI=0.187-0.898, FIG. 3A). In the 33 individuals with both tumor biopsies and PBMC pre-treatment samples (FIG. 3B), the number of shared clones was estimated based on having TCRs with identical CDR3 amino acid sequences and variable chains in tissue and PBMC. After normalizing for both tumor and PBMC sequencing, the presence of shared tumor and PBMC TCR clones was found to be significantly improved for overall survival (FIG. 3C, cox proportional hazard p-value=0.011). Patients with the top tertile of shared tumor biopsy and PBMC TCR clones had significantly better overall survival than patients with shared TCR clones in the bottom tertiles (p=0.012; HR=0.33, 95% CI=0.129-0.837).

Impact of On-Treatment TCR Clusters and Survival with Immunotherapy:

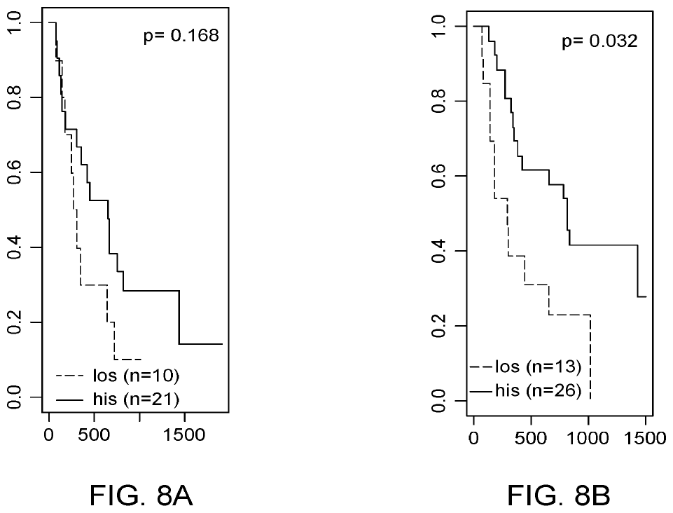

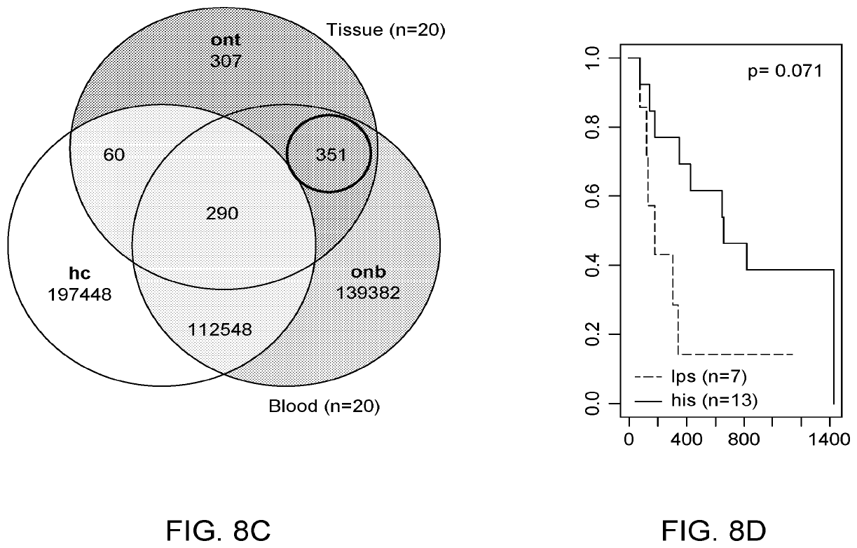

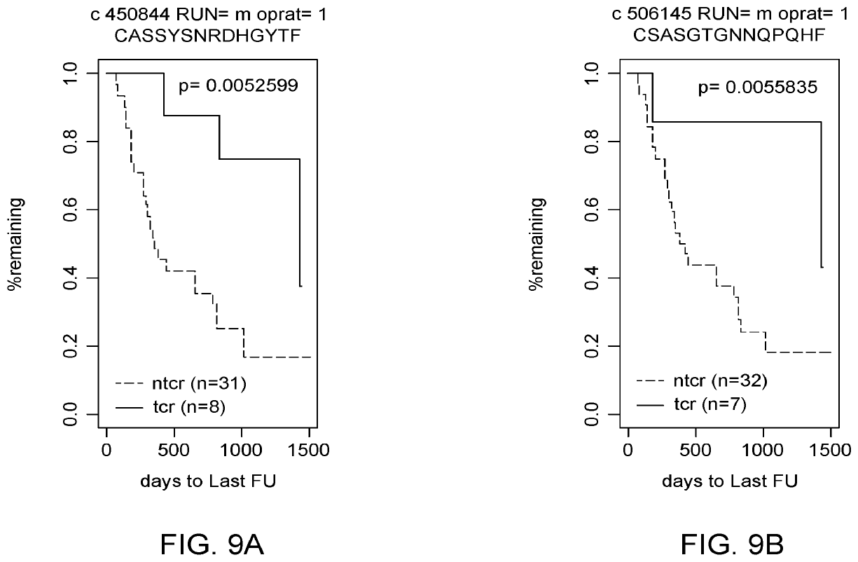

Similar trends were observed with TCR clusters detected from on-treatment tumor biopsies and PBMCs after administration of ICI (FIG. 8). Patients with TCR clusters in the top two tertiles from tumor biopsies or PBMCs had better overall survival than patients with TCR clusters in the bottom tertile. Additionally, patients with few shared TCR clones between tumor biopsies and PBMCs did poorly compared with other patients (FIG. 9). These findings suggested that patients with poor expansion of T cell repertoire or low number of T cells that trafficked to tumors were less likely to benefit from ICI.

Impact of Common TCR Clusters on Response and Survival with Immunotherapy:

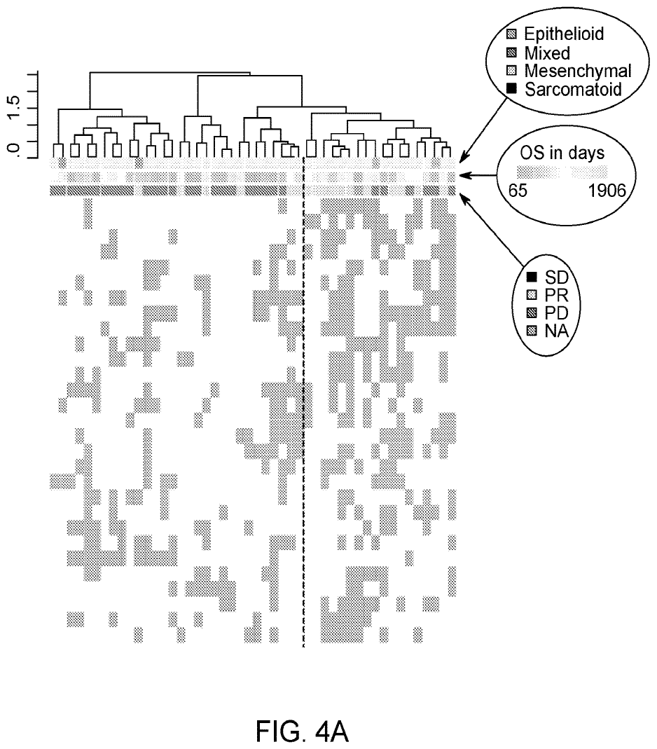

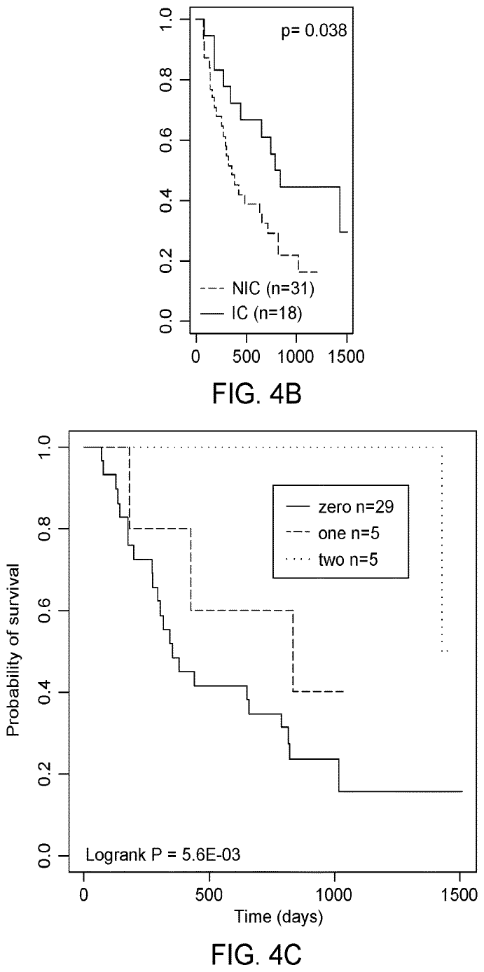

It was investigated whether membership in common mesothelioma TCR clusters in PBMCs had bearing on any clinical presentation. 29 TCR clusters were identified in the pre-treatment PBMCs that were each found in at least 12 patients. With the exception of one patient, all cases had TCR clones in at least 1 of these TCR clusters. In a heat map with columns representing individual PBMCs grouped by unsupervised clustering according to the dendrogram on top and rows representing TCR clusters, the data showed a group of patients that were enriched for commonly occurring TCR clusters (right side of the dashed line, FIG. 4A). This group that was enriched for commonly occurring TCR clusters contained a significantly higher proportion of patients with clinical responses to ICI (61% overall response rate according to modified pleural RECIST compared to 19% overall response rate, Chi-sq p=0.003) and better overall survival (HR=0.47, p=0.038; 95% CI=0.226-0.989; FIGS. 4A and 4B).

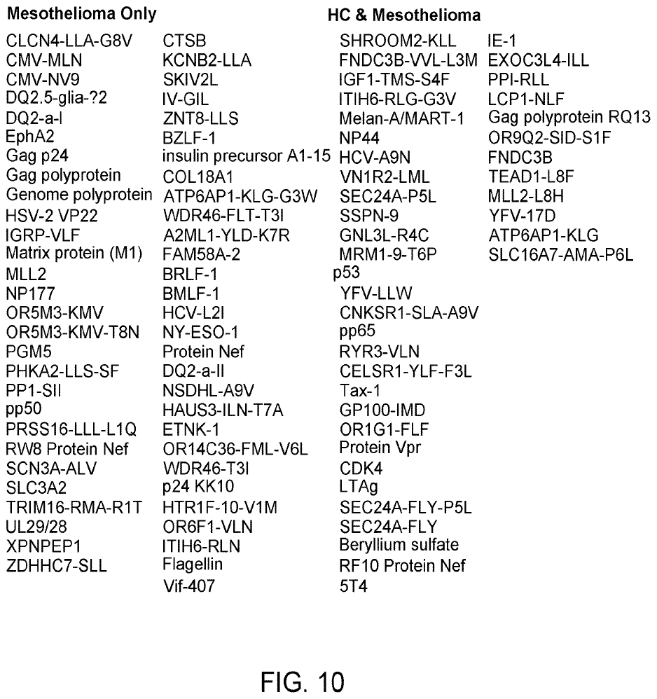

Association with survival outcome of TCR clusters in pre-treatment and on-treatment PBMCs was examined. None of the pre-treatment TCR clusters were associated with survival after correcting for FDR. In contrast, there were two TCR clusters in on-treatment PBMCs with FDR q values <0.06 (FIG. 6 and FIG. 10). There were five patients in which both of these TCR clusters were detected, and five patients in which one of these TCR clusters was detected. The detection of both TCR clusters provided significant survival benefit compared to detection of 1 cluster (HR<0.001, p=0.026) or the detection of no TCR clusters (HR=0.10, p=0.002; FIG. 4C). Whether these TCR clusters have been associated with known antigens, including mesothelin, was evaluated. Even though this search identified close to 100 known TCRs amongst all of the TCR clusters in the dataset, including 29 TCRs detected in only mesothelioma PBMCs (FIG. 10), the antigen specificity of these predictive TCR clusters is unknown.

Together, these results demonstrate that the presence of particular TCR clusters (e.g., a TCR that includes a CDR3 that includes an amino acid sequence set forth in SEQ ID NO:1 or SEQ ID NO:2) can be used to determine whether or not a mammal having mesothelioma is likely to respond to a particular cancer treatment (e.g., immunotherapy with one or more immune checkpoint inhibitors).

### Example 2: Survival Associations of T Cell Clusters with Immunotherapy in Mesothelioma

### Methods

TCR CDR3 was analyzed to identify unique clones. The unique clones were clustered to infer shared antigen specificity. The study cohort included patients with pleural mesothelioma who were treated with nivolumab (NivoMes, NCT02497508) or nivolumab and ipilimumab (INITIATE, NCT03048474) in second or later lines of therapy. TCR sequencing was performed with the ImmunoSEQ® assay (Adaptive Biotechnology) in 49 pre-treatment and 39 post-treatment patient peripheral blood mononuclear cell (PBMC) samples. These data were integrated with TCR sequences found in bulk RNAseq data by TRUST4 program in 42 pre-treatment and 31 on-treatment tumor biopsy samples and sequences from over 600 healthy controls (PMID 28369038). Geometric Isometry-based TCR AligNment Algorithm (GIANA) (PMID 31831563) was used to identify TCR clusters. Associations of TCR clusters with overall survival (OS) were determined by cox proportional hazard analysis.

### Results

4.2 million and 12 thousand CDR3 sequences from PBMCs and tumors, respectively, were identified in patients treated with ICBT. The CDR3 sequences were integrated with 2.1 million publically available CDR3 sequences from healthy controls and clustered. Cluster sizes ranged from 2 to about 3900 CDR3 sequences, with the majority of clusters having 2-3 CDR3s (FIG. 1B). To select anti-tumor clusters, the study filtered for clusters that were (1) not found in healthy controls (2) recurrent in multiple patients with mesothelioma, and (3) more prevalent in post-treatment samples than pre-treatment samples.

This approach identified 29 CDR3 clusters among which two clusters were significantly associated with OS following ICBT (Hazard Ratios 0.17-0.23, p values all <0.006). These clusters were not found in bulk tissue RNA-seq data and were not found as reported in public CDR3 databases.

The CDR3 amino acid (aa) sequences of cluster 450844 are shown in Table 2. The CDR3 aa sequences of cluster 506145 are shown in Table 3. Patients having T cells with cluster 450844 TCRs 450844 TCRs, cluster 506145 TCRs, or both had significantly improved survival compared to patients that lacked either (FIG. 4C).

TABLE 2
TCR CDR3 sequence cluster 450844.

| CDR3 amino acid | SEQ | | | | | |
|---|---|---|---|---|---|---|
| sequence | ID NO | clusterId | V segment | Pre/On | oo | pr |
| CASSYSDQDYGYTF | 3 | 450844 | TRBV6-5*01 | On | 8 | 1 |
| CASSYSDRDYGYTF | 4 | 450844 | TRBV6-5*01 | On | 12 | 1 |
| CASSYSNRDYGYTF | 5 | 450844 | TRBV6-5*01 | On | 2 | 1 |
| CASSYSSRDFGYTF | 6 | 450844 | TRBV6-5*01 | On | 3 | 1 |
| CASSYSSRDYGYTF | 7 | 450844 | TRBV6-5*01 | On | 4 | 1 |
| CASSYSSRDYGYTF | 7 | 450844 | TRBV6-5*01 | On | 5 | 1 |
| CASSYSSRDYGYTF | 7 | 450844 | TRBV6-6*01 | On | 6 | 1 |
| CASSYSSRDYGYTF | 7 | 450844 | TRBV6-6*01 | On | 7 | 1 |
| CASSYSDRDYGYTF | 4 | 450844 | TRBV6-2*01 | Pr | 9 | 0 |
| CASSYSDRDYGYTF | 4 | 450844 | TRBV6-5*01 | Pr | 10 | 0 |
| CASSYSDRDYGYTF | 4 | 450844 | TRBV6-5*01 | Pr | 11 | 0 |
| CASSYSDRDYGYTF | 4 | 450844 | TRBV6-6*01 | Pr | 13 | 0 |
| CASSYSERDYGYTF | 8 | 450844 | TRBV6-6*01 | Pr | 14 | 0 |
| CASSYSNRDHGYTF | 9 | 450844 | TRBV6-5*01 | Pr | 1 | 0 |

TABLE 3
TCR CDR3 sequence cluster 506145.

| CDR3 amino acid | SEQ | | | | | |
|---|---|---|---|---|---|---|
| sequence | ID NO | clusterId | V segment | | oo | pr |
| CSASGTGANQPQHF | 10 | 506145 | TRBV20-1*01 | On | 7 | 1 |
| CSASGTGNNQPQHF | 11 | 506145 | TRBV20-1*01 | On | 1 | 1 |
| CSASGTGSNQPQHF | 12 | 506145 | TRBV20-1*01 | On | 2 | 1 |
| CSASGTGSNQPQHF | 12 | 506145 | TRBV20-1*01 | On | 3 | 1 |
| CSATGTGSNQPQHF | 13 | 506145 | TRBV20-1*01 | On | 8 | 1 |
| CSATGTGSNQPQHF | 13 | 506145 | TRBV20-1*01 | On | 10 | 1 |
| CSVSGTGSNQPQHF | 14 | 506145 | TRBV20-1*04 | On | 4 | 1 |
| CSATGTGNNQPQHF | 15 | 506145 | TRBV20-1*01 | Pr | 6 | 0 |
| CSATGTGSNQPQHF | 13 | 506145 | TRBV20-1*01 | Pr | 9 | 0 |
| CSASGTGAHQPQHF | 16 | 506145 | TRBV20-1*04 | Pr | 5 | 0 |

These clusters can be used to improve patient selection for immunotherapy, enable approaches for antigen discovery and inform future targets for design of adoptive T cell therapies.

### Example 3: Treating Pleural Mesothelioma

A blood sample containing PBMCs is obtained from a human having pleural mesothelioma. The obtained sample is examined to determine whether T cells have the presence or absence of a TCR including a CDR3 that includes the amino acid sequence set forth in SEQ ID NO:1 or SEQ ID NO:2.

If the presence of a TCR including a CDR3 that includes the amino acid sequence set forth in SEQ ID NO: 1 or SEQ ID NO:2 is detected in one or more T cells within the sample, then the human is identified as being likely to respond to one or more immune checkpoint inhibitors, and the human is administered one or more immune checkpoint inhibitors.

The administered one or more immune checkpoint inhibitors can reduce the number of cancer cells within the human while improving the survival of the human.

### Example 4: Treating Pleural Mesothelioma

A blood sample containing PBMCs is obtained from a human having pleural mesothelioma. The obtained sample is examined to determine whether T cells have a TCR that includes a CDR3 that includes the amino acid sequence set forth in SEQ ID NO:1 or SEQ ID NO:2 in one or more T cells within the sample.

If TCRs that include a CDR3 that includes the amino acid sequence set forth in SEQ ID NO: 1 or SEQ ID NO:2 are not detected in T cells within the sample, then the human is identified as not being likely to respond to one or more immune checkpoint inhibitors, and the human is instead administered one or more alternative cancer treatments.

The administered alternative cancer treatments can reduce number of cancer cells within the human while improving the survival of the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-8. (canceled)

9. A method for treating a mammal having pleural mesothelioma, wherein said method comprises:
(a) determining that said mammal comprises (i) T cells having a TCR comprising a CDR3 comprising the amino acid sequence set forth in SEQ ID NO:1 or (ii) T cells having a TCR comprising a CDR3 comprising the amino acid sequence set forth in SEQ ID NO:2; and
(b) administering an immune checkpoint inhibitor to said mammal.

10. The method of claim 9, wherein said mammal is a human.

11. The method of claim 9, wherein a blood sample obtained from said mammal is used to determine that said mammal comprises said (i) or (ii).

12. The method of claim 11, wherein said blood sample comprises PBMCs.

13. The method of claim 9, wherein said mammal comprises each of said (i) and (ii).

14. The method of claim 9, wherein said method comprises administering to said mammal a cancer treatment selected from the group consisting of cisplatin, pemetrexed, surgery, radiation therapy, and ablative therapy.

15-21. (canceled)

22. A method for treating pleural mesothelioma, wherein said method comprises administering an immune checkpoint inhibitor to a mammal having pleural mesothelioma that was determined to comprise (i) T cells having a TCR comprising a CDR3 comprising the amino acid sequence set forth in SEQ ID NO:1 or (ii) T cells having a TCR comprising a CDR3 comprising the amino acid sequence set forth in SEQ ID NO:2.

23. The method of claim 22, wherein said mammal is a human.

24. The method of claim 22, wherein a blood sample obtained from said mammal was used to determine that said mammal comprises said (i) or (ii).

25. The method of claim 24, wherein said blood sample comprises PBMCs.

26. The method of claim 22, wherein said mammal was determined to comprise each of said (i) and (ii).

27. The method of claim 22, wherein said method comprises administering to said mammal a cancer treatment selected from the group consisting of cisplatin, pemetrexed, surgery, radiation therapy, and ablative therapy.

28. A method for treating mesothelioma, wherein said method comprises administering a cancer treatment to a mammal having mesothelioma that was determined to not comprise (i) T cells having a TCR comprising a CDR3 comprising the amino acid sequence set forth in SEQ ID NO:1 and (ii) T cells having a TCR comprising a CDR3 comprising the amino acid sequence set forth in SEQ ID NO:2, wherein said cancer treatment is not an immune checkpoint inhibitor.

29. The method of claim 28, wherein said mammal is a human.

30. The method of claim 28, wherein a blood sample obtained from said mammal was used to determine that said mammal does not comprise said (i) and (ii).

31. The method of claim 30, wherein said blood sample comprises PBMCs.

32. The method of claim 28, wherein said mesothelioma is a pleural mesothelioma.

33. The method of claim 28, wherein said cancer treatment comprises administering to said mammal a cancer drug selected from the group consisting of cisplatin, pemetrexed, carboplatin, and bevacizumab.

34. The method of claim 28, wherein said cancer treatment comprises subjecting said mammal to a treatment selected from the group consisting of surgery, radiation therapy, and ablative therapy.


SEQUENCE LISTING (16 sequences)

SEQ ID NO: 1 - protein, 14 aa, synthetic construct
  VARIANT 7: note = X can be N, S, D, or E
  VARIANT 8: note = X can be R or Q
  VARIANT 10: note = X can be H, Y, or F
CASSYSXXDXGYTF

SEQ ID NO: 2 - protein, 14 aa, synthetic construct
  VARIANT 3: note = X can be A or V
  VARIANT 4: note = X can be S or T
  VARIANT 8: note = X can be N, S, or A
  VARIANT 9: note = X can be N or H
CSXXGTGXXQPQHF

SEQ ID NO: 3 - protein, 14 aa, Homo sapiens
CASSYSDQDYGYTF

SEQ ID NO: 4 - protein, 14 aa, Homo sapiens
CASSYSDRDYGYTF

SEQ ID NO: 5 - protein, 14 aa, Homo sapiens
CASSYSNRDYGYTF

SEQ ID NO: 6 - protein, 14 aa, Homo sapiens
CASSYSSRDFGYTF

SEQ ID NO: 7 - protein, 14 aa, Homo sapiens
CASSYSSRDYGYTF

SEQ ID NO: 8 - protein, 14 aa, Homo sapiens
CASSYSERDYGYTF

SEQ ID NO: 9 - protein, 14 aa, Homo sapiens
CASSYSNRDHGYTF

SEQ ID NO: 10 - protein, 14 aa, Homo sapiens
CSASGTGANQPQHF

SEQ ID NO: 11 - protein, 14 aa, Homo sapiens
CSASGTGNNQPQHF

SEQ ID NO: 12 - protein, 14 aa, Homo sapiens
CSASGTGSNQPQHF

SEQ ID NO: 13 - protein, 14 aa, Homo sapiens
CSATGTGSNQPQHF

SEQ ID NO: 14 - protein, 14 aa, Homo sapiens
CSVSGTGSNQPQHF

SEQ ID NO: 15 - protein, 14 aa, Homo sapiens
CSATGTGNNQPQHF

SEQ ID NO: 16 - protein, 14 aa, Homo sapiens
CSASGTGAHQPQHF